(19) United States
(12) Patent Application Publication
(10) Pub. No.: US 2005/0119697 A1
(43) Pub. Date: Jun. 2, 2005

(54) Method of table mounted retraction in hip surgery

(75) Inventor: Todd William Sharratt, Birchwood, MN (US)

Correspondence Address: Z. Peter Sawicki;Westman, Champlin & Kelly, Suite 1600, 900 Second Avenue South, Minneapolis, MN, 55402-3319, US

(73) Assignee: Minnesota Scientific, Inc., St. Paul, MN (US)

(21) Appl. No.: 11/032,548

(22) Filed: Jan. 10, 2005

Related U.S. Application Data
(63) Continuation-in-part of application No. 10/728,202, filed on Dec. 4, 2003.
Application No. 10/728,202 is a continuation of application No. 10/077,693, filed on Feb. 15, 2002, now Pat. No. 6,659,944.
Application No. 10/077,693 is a continuation of application No. 09/990,420, filed on Nov. 21, 2001, now Pat. No. 6,368,271.
(63) Continuation-in-part of application No. 10/892,816, filed on Jul. 16, 2004.
Application No. 10/892,816 is a continuation-in-part of application No. 10/623,179, filed on Jul. 18, 2003.
(60) Provisional application No. 60/396,850, filed on Jul. 18, 2002.

(51) Int. Cl. A61L 17/00

(52) U.S. Cl. 606/228

(57) ABSTRACT

A method of performing a hip joint surgery on a patient positioned on a surgical table where the hip joint includes a pelvis having an acetabulum, a femur and a femoral ball includes mounting a retractor support to the surgical table. The retractor support is positioned about the hip joint and about an incision into the skin and flesh layers proximate the hip joint. A retractor is positioned within the incision and is manually retracted to retract skin and flesh layers proximate the hip joint. The retractor is secured in a selected position by attaching the retractor to the retractor support.

### CROSS REFERENCE TO RELATED APPLICATIONS

This application is a continuation-in-part of application Ser. No. 10/728,202 filed on Dec. 4, 2003, which is hereby incorporated by reference in its entirety, which is a continuation of application Ser. No. 10/077,693, filed Feb. 15, 2002, which is incorporated by reference in its entirety, and resulting in U.S. Pat. No. 6,659,944, which is a continuation of application Ser. No. 09/990,420 filed on Nov. 21, 2001, which is incorporated by reference in its entirety, and resulting in U.S. Pat. No. 6,368,271.

This application is also a continuation-in-part of application Ser. No. 10/892,816 filed on Jul. 16, 2004, which is hereby incorporated by reference in its entirety, which is a continuation-in-part of application Ser. No. 10/623,179; filed Jul. 18, 2003, which is hereby incorporated by reference in its entirety, which claims priority of U.S. Provisional Application No. 60/396,850, filed Jul. 18, 2002, the content of which is hereby incorporated by reference in its entirety.

### BACKGROUND OF THE INVENTION

The present invention relates to a method of surgical retraction. In particular, the present invention relates to a method of utilizing a table mounted retracting device during a surgical procedure on a hip of a patient.

Total hip replacement (arthroplasty) operations have been performed since the early 1960's to repair hip components. These components include the acetabulum (socket portion of the hip) and the femoral head (ball portion of the hip). The hip is typically replaced due to a gradual deterioration of the cartilage that cushions the bones within the joint. The surrounding structures in the hip can become inflamed and painful. Eventually, bone can begin to rub against bone causing severe discomfort.

Surgical procedures have been the most successful method to alleviate this pain. Either partial or total hip replacement surgery can be used. In total hip replacement, a cup shaped insert typically manufactured of polyethylene is inserted in place of the acetabulum, and a metal femoral head is placed in the femur. A number of variations have evolved in the surgical approaches and techniques used for replacement of the hip components, including operating while the patient is on his or her back (supine) or on his or her side (lateral). To a large extent, the choice of surgical approaches is due to the surgeon's preference as to what aspect of the hip components the surgeon wishes to view. The ability to view the surgical site is complicated by the need to remove the femoral head from the acetabulum as well as rotate and retract the femur in the wound during surgery.

The surgical procedure can become quite physically taxing on the surgeon or surgeons performing it. The surgical procedure requires lifting and moving the patient's femur into multiple positions. At times, the surgeon may need to hold the femur in position for an extended period of time. Depending on the size of the patient, the strenuous activity can lead to fatigue and contribute to surgical error. Additionally, the repeated movement of the leg can cause nerve damage if it is not done precisely and with minimal adjustment. When the surgeon moves the femur by hand it is common to have continual adjusting occur. Often, the surgeon holding the leg relaxes or becomes fatigued and allows the leg to move, requiring that the leg be readjusted. The movement can cause the leg to pinch or rub nerves or muscle tissue, possibly causing damage.

One way to reduce the physical nature of the operation and the number of personnel required to perform the procedure is to use retractors secured to a support that is secured to a surgical table to retract the flesh to expose the surgical site. U.S. Pat. No. 6,315,718 discloses a table mounted retractor system for a method of hip retraction. The table mounted retractor system includes using a table mounted support apparatus to support both flesh retracting retractors to expose the hip joint and bone retracting retractors to dislocate and displace the femural ball from the acetabulum.

### SUMMARY OF THE INVENTION

The present invention includes a method of performing a hip joint surgery on a patient positioned on a surgical table where the hip joint includes a pelvis having an acetabulum, a femur and a femoral ball. The method includes mounting a retractor support to the surgical table. The retractor support is positioned about the hip joint and about an incision into the skin and flesh layers proximate the hip joint. A retractor is positioned within the incision and is manually retracted to retract skin and flesh layers proximate the hip joint. The retractor is secured in a selected position by attaching the retractor to the retractor support.

### BRIEF DESCRIPTION OF THE DRAWINGS

FIG. 1 is a top view of a surgical support apparatus positioned about a hip joint;

FIG. 2 is a top view of a surgical support apparatus retracting a femur from an acetabulum in a pelvis;

FIG. 3 is a top view of a surgical support apparatus wherein a prosthetic insert is inserted into the femur in the acetabulum of the pelvis; and

FIG. 4 is a perspective view of a clamp having a clamping socket for securing a retractor handle therein.

### DETAILED DESCRIPTION OF THE PREFERRED EMBODIMENTS

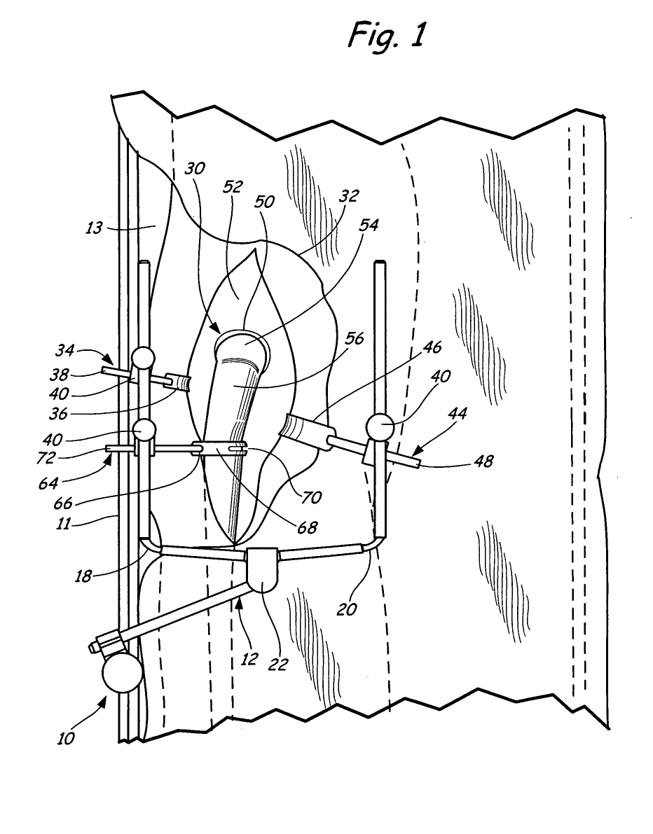

The present invention relates to a method of performing surgical procedures on or proximate a hip joint. An apparatus used in the surgical procedures of the present invention is generally indicated at 10 in FIG. 1.

The apparatus 10 includes a retractor support apparatus 12 that is rigidly mounted to a rail 11 of a surgical table 13 in a manner that is well known in the art and is described in U.S. Pat. Nos. 4,617,916, 4,718,151, 4,949,707, 5,400,772, 5,741,210, 6,042,541, 6,264,396 and 6,315,718 all of which are herein incorporated by reference. From the mount to the surgical table 13, the retractor support apparatus 12 includes left and right support arms 18 and 20 that extend over the surgical table 13. The support arms 18 and 20 are independently adjustable into an infinite number of selected positions through use of a clamping mechanism 22 which is described in U.S. Pat. Nos. 5,899,627 and 6,264,396, which are herein incorporated by reference. The support arms 18 and 20 extend in a generally lateral or horizontal direction on opposite sides of a hip joint 30. The clamp 22 secures the adjustable support arms 18 and 20 in selected angular positions with respect to the hip joint 30.

The surgical procedure on the hip joint 30 can be performed with any table mounted support structure and is not limited by the configuration illustrated and described herein. However, the table mounted support structure must provide support for mounting retractors to retract skin, muscle, blood vessels, tendons and bone while providing access to the hip joint 30.

With the table mounted support structure 12 positioned in a selected position about the hip joint 30, an incision 32 is made through the skin. Typically, the incision is about six to eight inches long but may vary depending upon the surgical procedure being performed.

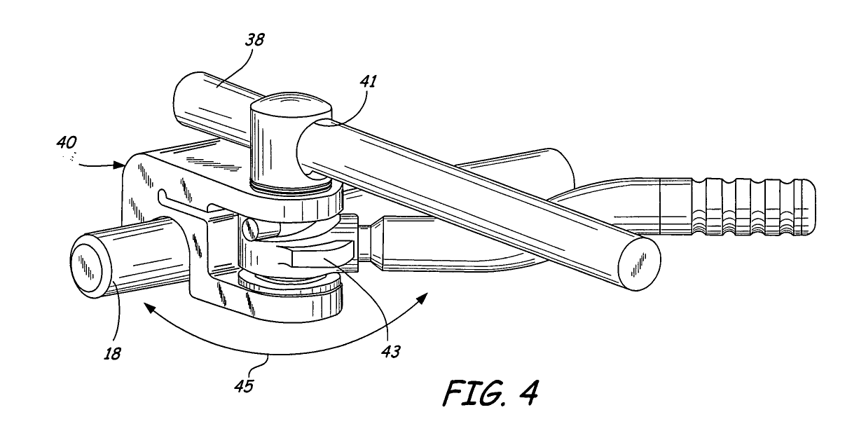

After making the incision 32 through the skin, the surgeon divides the tissue, muscle, blood vessels and nerves to expose the hip joint 30 while causing minimal trauma. To expose the hip joint 30, a retractor blade 36 of a lateral retractor 34 is positioned within the incision 32. The tissue, muscle, blood vessels and nerves are laterally retracted from the incision with manual force applied to the lateral retractor 34. With the lateral retractor 34 in the selected position, a handle 38 of the lateral retractor 34 is positioned within a clamping socket 41 of a clamp 40 positioned on the support arm 18. The lateral retractor is secured in the selected position by positioning the clamp 40 in the clamping position by rotating a clamping mechanism 43 in the direction of arrows 45. The clamp 40 having the clamping socket 41 is illustrated in FIG. 4.

Preferably, the clamping socket 41 allows the retractor handle 38 to be manually forced therein without having to position an end of the handle 38 through the clamping socket 42, although other clamps, including clamps with clamping bores are within the scope of the present invention. By socket is meant an opening or a cavity into which an inserted part, such as a retractor support apparatus, is designed to fit and wherein the retractor support apparatus can be inserted into the socket from an infinite number of directions in a 180° range starting from a substantially parallel position to a back surface of the socket to a position substantially perpendicular to the back surface and continuing to position again substantially parallel to the back surface of the socket. By lateral is meant a position or direction generally away from the body.

A retractor blade 44 of a medial retractor 42 is positioned within the incision 32 generally opposite the lateral retractor 34. Manual force is applied the medial retractor 42 to retract tissue, muscle, blood vessels and nerves in a medial direction from the hip joint 30. With the medial retractor 42 in a selected position, a handle 46 of the medial retractor 42 is positioned within a clamping socket 41 of a clamp 40 and the clamp 40 is positioned on the support arm 20. The clamp 40 is identical to the clamp 40 used to secure the lateral retractor 34 in the selected position. The clamp 40 is secured in the selected position by rotating the clamping mechanism 43 in a direction of arrows 45 as illustrated in FIG. 4 and thereby positioning the clamp 40 into the clamping position. By medial is meant a position or direction toward the body of the patient.

Although two retractors 34, 44 are a preferred number of retractors for exposing the surgical site about the hip, more than two retractors supported by the table mounted retractor support apparatus 12 can be utilized to expose the surgical site in the hip while practicing the surgical procedure of the present invention.

Mechanical mechanisms (not shown) on retractors may be used to adjust the vertical position of the retractor blade and the lateral or medial position of the retractor blade within the surgical site. Mechanical mechanisms used to adjust the position of the retractor blade include, but are not limited to, articulated joints, rack and pinion systems, racheting mechanisms, wedges, ramps, camming mechanisms and threadable engagements.

With the hip joint 30 exposed by the retraction of the skin, tissue, muscle, blood vessels and nerves, the surgical procedure on the hip can be performed. A non-exhaustive list of surgical procedures that can be performed on the hip include repair of a muscle tear, repair of a torn or ruptured tendon or ligament, as well as a hip replacement surgery. The hip replacement surgery includes a complete hip replacement and a partial hip replacement surgery.

In a hip replacement surgery, the skin, muscle, blood vessels and nerves are preferably retracted, without having to be severed with a scalpel, to expose the hip joint 30 with at least the lateral and medial retractors. Performing the surgery on the hip joint 30 while minimizing the damage the muscle, blood vessels and nerves minimizes the post-operative pain felt by the patient as well as reducing the time required to rehabilitate the hip joint. However, it is within the scope of the present invention to perform a surgical procedure that requires incising muscles, blood vessels and nerves around the hip joint, if necessary.

With the hip joint exposed, the acetabulum 50 within a pelvis 22, the femoral ball 54 and an upper portion of the femur 56 are viewable through the retracted incision 32. In preparing the hip joint 30 for the hip replacement surgery, the surgeon has two options in gaining access to the surfaces that accept the inserts. First, the femoral ball 54 may be separated from the femur 56, typically with a bone saw, while the femoral ball 54 remains within the acetabulum 50.

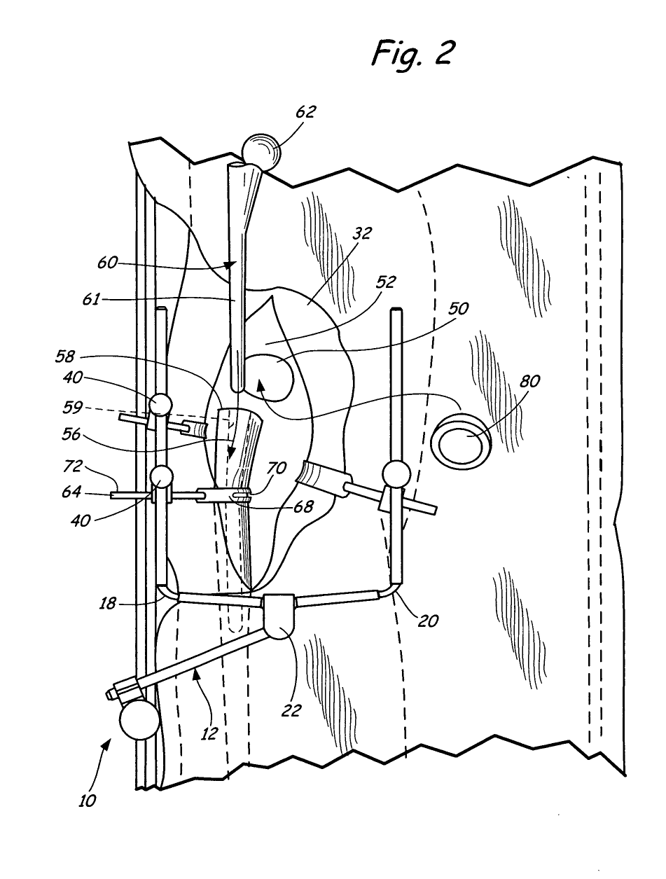

Referring to FIG. 2, the femur 56 is retracted laterally away from the hip joint 30 to gain access to a freshly cut surface 58 on the femur 56 onto which a metal or ceramic femoral insert having a ball 62. The femur 56 is retracted with a retractor 64 having a blade 66 similar to a Fakuda blade, which is known in the art. The blade includes a generally flat portion 68 and an arcuate end (not shown) that is positioned about the upper portion of the femur 56. The blade also includes an aperture 70 within both the generally flat portion 68 and the arcuate end (not shown) that aids in gripping the femur 56 and prevents the femur 56 from slipping-along the blade 66. However, the aperture 70, while providing additional gripping capability to the retractor blade 66, is not necessary to retract the. femur 56 away from the acetabulum 50.

The femur 56 is retracted with manual force placed upon the retractor 64. With the femur 56 manually retracted into a selected position, the retractor 64 is clamped to the support arm 18 by positioning a retractor handle 72 into a clamping socket 42 of a clamp 40 and positioning the clamp 40 into the clamping position. Alternatively, the femur can be retracted with a retractor blade attached to a retractor blade holder supported by the retractor support apparatus 12 where the vertical position is adjusted with an articulated joint and the femur is retracted with a rack and pinion mechanism on the retractor blade holder.

The femur 56 is retracted from the acetabulum 50 a selected distance to provide the surgeon access to both a freshly cut surface 58 on the femur 56 and also to the acetabulum 50 having the detached femoral ball 54 retained therein. The detached femoral ball 54 is dislocated from the acetabulum 50 to gain access to the acetabulum 50 for preparation to accept an insert 80.

Alternatively, the femoral ball 54, while attached to the femur 56, may be first dislocated from the acetabulum 50 to gain access to the acetabulum 50 for preparation to accept the insert 86. The femur 56 is laterally retracted, either manually or with a mechanical mechanism as previously disclosed, from the hip joint 30 to gain access to both the femoral ball 54 and the acetabulum 50. The femoral ball 54 is then separated from the femur 56, typically with a bone saw, thereby creating the surface 58 to which the insert 60 is secured.

The femoral insert 60 that replaces the femoral ball 56 can be shaped to conform to the cut surface 58 of the femur 56 and cemented into place. The insert 60 may also include a single shaft 61 that is positioned within a cooperating cavity 59 in the femur 56. Alternatively, a plurality of pegs (not shown) extending from the insert 60 are inserted into cooperating cavities (not shown) that are reamed into the femur 56. The insert 60 is then cemented into place.

Alternatively, the insert 60 may include mesh-like surface (not shown) may be positioned onto the femur 56 or within the cavity 59 reamed into the femur 52 such that mesh-like surfaces engage the femur 56 and allows the bone to grow onto the insert 60 to secure the insert 60 to the femur 56. The femoral insert 60 is preferably constructed of a highly polished metal such as stainless steel or titanium or a ceramic material.

After the end of the femur 56 has been prepared to accept the prosthetic insert 60, the acetabulum 50 is also prepared to accept the second prosthetic insert 80 by reaming a pelvis 52 to a selected configuration similar to the outer surface of the insert 80. One embodiment of the insert 80 that is positioned within the acetabulum 50 is constructed from a high density polymer, such as polyethylene, which interacts with the polished femoral insert 60 to reconstruct the hip joint 30. Another embodiment of the insert 80 includes two components, a highly polished metal component (not shown) that is secured to the pelvis 52 having the acetabulum 50 and a polymer component (not shown) that is secured to the metal component (not shown) where the polymer component engages the femoral insert 60. The metal component (not shown) may include a shaft or a plurality of pegs that engage complimentary cavities that are reamed into the pelvis 52 that includes the acetabulum where the metal component is cemented to the pelvis. Alternatively, the metallic portion insert may include a mesh-like surface that is positioned within the acetabulum where the pelvis grows onto the insert to secure the metallic portion of the insert to the bone.

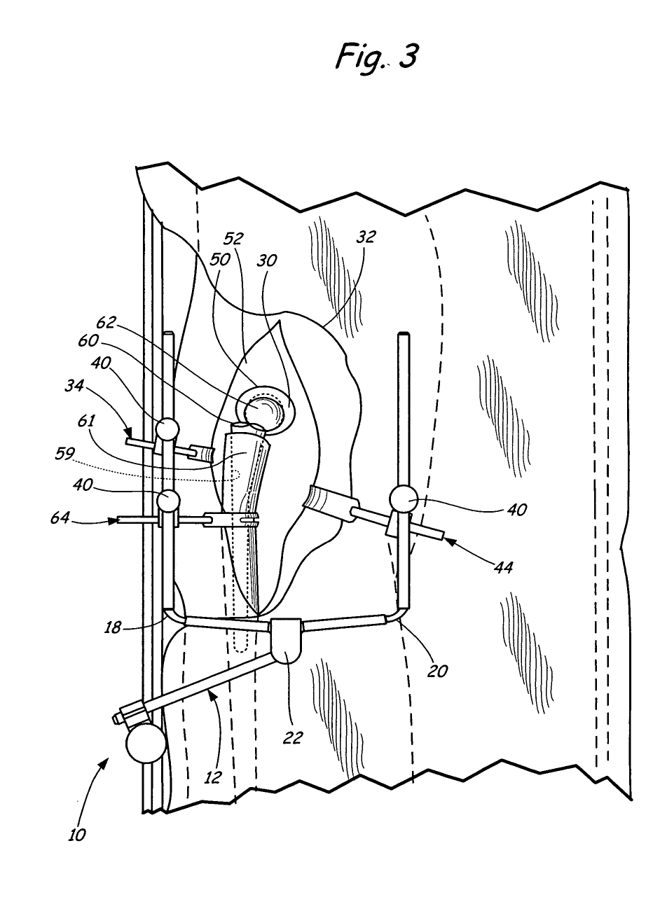

Although either insert 80 is within the scope of the present invention, the two component insert provides an advantage of replacing only the polymeric portion of the insert without having to perform additional surgery on the pelvis in the event the polymeric portion of the insert wears and causes the patient discomfort. With the prosthetic insert 80 secured to the pelvis 52 within the acetabulum 50, the femoral insert 60 is reducted into the insert 80 within the acetabulum 50 as illustrated in FIG. 3.

One skilled in the art will recognize that in a total hip-joint replacement surgery, all of the cartilage and synovial membrane attached to or positioned between the pelvis and the femur are removed, which provides the nearly frictionless interaction between the femoral ball and the acetabulum when undamaged. However, the highly polished insert 60 attached to the femur 56 engages the high density polymer insert 80 attached to the pelvis 52 within the acetabulum 50 such that the interaction of the inserts 60, 80 is almost frictionless and resembling the function of a healthy hip joint 30.

Although the total hip-joint replacement surgery described first prepares the femur 56 for the femoral insert 60 and then prepares the acetabulum 50 for the pelvic insert 80, it is within the scope of the present invention to first prepare the acetabulum 50 followed by the femur 56 for accepting prosthetic inserts 80, 60, respectively. Additionally, it is within the scope of the present invention to replace the damaged end of either the femur 56 or the acetabulum 50 in the pelvis 52 with an insert while leaving the undamaged end of the other bone intact.

Once the inserts 60, 80 have been secured to the femur 56 and within the acetabulum 50 within the pelvis 56, respectively, the lateral and medial retractors 34, 44 are removed from the incision 32 and the incision 32 is sutured closed. A drain (not shown) may be positioned within the hip joint 30 to remove excess blood and fluids that may accumulate in the hip joint 30 caused by the trauma from the total or partial hip joint replacement surgery. Once the hip joint 30 stops draining, the drain is removed and the incision 32 is completely closed.

Although the present invention has been described with reference to preferred embodiments, workers skilled in the art will recognize that changes may be made in form and detail without departing from the spirit and scope of the invention.

1. A method of performing a hip joint surgery on a patient positioned on a surgical table, the hip joint comprising a pelvis having an acetabulum, a femur and a femorral ball, the method comprising:
mounting a retractor support to the surgical table;
positioning the retractor support about the hip joint;
incising skin and flesh layers proximate the hip joint;
manually retracting the skin and flesh layers proximate the hip joint with a retractor; and
securing the retractor in a selected retracting position by attaching the retractor to the retractor support.

2. The method of claim 1 and wherein the retractor support is mounted to a rail of the surgical table.

3. The method of claim 1 and wherein the hip joint surgery comprises a hip joint replacement surgery wherein an insert is secured within the acetabulum and a femoral insert is secured to the femur.

4. The method of claim 1 and wherein the hip joint surgery comprises a partial hip joint surgery wherein a femoral insert is secured to the femur.

5. The method of claim 1 and further comprising:
dislocating the femoral ball from the acetabulum;
positioning a femur retractor about the femur;
manually retracting the femoral ball from the acetabulum; and
securing the femur retractor to the retractor support.

6. The method of claim 1 and further comprising separating the femoral ball from the femur with the femoral ball remaining in the acetabulum.

7. The method of claim 6 and further comprising removing the femoral ball from the femur and thereby exposing a freshly cut surface.

8. The method of claim 6 and further comprising:
reaming a cavity into a freshly cut surface of the femur;
positioning a stem having a ball attached thereto into the cavity in the femur; and
securing the stem within the cavity such that the ball is fixed into a selected position on the femur.

9. The method of claim 6 and further comprising:
dislocating the femoral ball from the acetabulum;
preparing the acetabulum within the pelvis to accept an insert; and
securing an insert to the pelvis and within the acetabulum.

10. The method of claim 9 and further comprising disposing the ball attached to the femur within the insert secured within the acetabulum.

11. A method of preparing a surgical site for a surgical procedure on a hip joint, the method comprising:
mounting a retractor support to the surgical table;
positioning the retractor support about the hip joint;
incising skin and flesh layers proximate the hip joint;
manually retracting the skin and flesh layers proximate the hip joint with a retractor to expose a surgical site; and
attaching the retractor to the retractor support.

12. The method of claim 11 and wherein the retractor support is mounted to a rail of the surgical table.

13. The method of claim 11 and wherein the surgical procedure comprises a hip joint replacement surgery wherein an insert is secured within the acetabulum and a femoral insert is secured to the femur.

14. The method of claim 11 and wherein the surgical procedure comprises a partial hip joint surgery wherein a femoral insert is secured to the femur.

15. The method of claim 11 and wherein the surgical procedure comprises repairing a damaged muscle, tendon or ligament.
